(19) United States
(12) Patent Application Publication
(10) Pub. No.: US 2025/0268914 A1
(43) Pub. Date: Aug. 28, 2025

(54) FLUTICASONE DELIVERY COMPOSITIONS, DEVICES AND METHODS

(71) Applicant: HONEYWELL INTERNATIONAL INC., Charlotte, NC (US)

(72) Inventors: Erik Boldt, Buffalo, NY (US); Gregory Smith, Buffalo, NY (US)

(73) Assignee: HONEYWELL INTERNATIONAL INC., Charlotte, NC (US)

(21) Appl. No.: 19/057,509

(22) Filed: Feb. 19, 2025

Related U.S. Application Data
(60) Provisional application No. 63/558,084, filed on Feb. 26, 2024.

(51) Int. Cl. A61K 31/56; A61K 9/10; A61K 47/10

(52) U.S. Cl. CPC A61K 31/56; A61K 9/10; A61K 47/10

(57) ABSTRACT

Disclosed are medicinal compositions, and devices, methods and systems which use same, comprising a carrier and at least one medicinally active compound, said carrier comprising at least 1234ze(E) and ethanol.

### CROSS REFERENCE TO RELATED APPLICATION

This application is related to and claims the priority benefit of U.S. Provisional Application No. 63/558,084, filed Feb. 26, 2024, which is incorporated by reference herein in its entirety.

### FIELD OF THE INVENTION

This invention relates to medicament delivery compositions, systems, devices and methods. Particular aspects of the invention relate to medicinal aerosol compositions, methods and devices used for metered dose delivery of fluticasone.

### BACKGROUND OF THE INVENTION

Metered dose inhalers (MDIs) have long been used for oral and/or nasal delivery of medicaments, such as inhaled corticosteroids (ICSs), to a patient in need of treatment. Compared with oral administration, inhalation therapy using MDIs frequently has the advantage of relatively rapid onset of action and relatively low instance of systemic side effects.

MDIs may be used to deliver medicaments in a solubilized form or as a suspension. Typically, MDIs use a relatively high vapor pressure propellant to carry and expel aerosolized droplets containing an API (sometimes referred to herein for convenience as “pMPI”) into the respiratory tract when the pMDI is activated. The propellant/carrier for the active pharmaceutical ingredient (sometimes referred to herein as “API”) must be safe for patients' use and be pharmaceutically acceptable. The API to be delivered by a pMDI is typically provided as a suspension of particulates dispersed within a carrier, and one function of the carrier is to help form a suspension of and/or carry the API.

Several challenges exist in connection with efforts to provide new pMDIs in general, and suspension-based pMDI propellants in particular. For example, while there are advantages to delivery of the API as fine particles suspended in a carrier, there is a tendency of such particles of API to aggregate and/or flocculate. This problem can have an impact on the effectiveness of use of the pMDI.

An important objective of suspension pMDIs is to accurately deliver, upon actuation by the person in need of relief, a specific predetermined amount of a drug to the respiratory tract of a patient using a delivery composition in which the drug is suspended or dispersed. The delivery composition will generally include, in addition to the API, a propellant and potentially components which aid in the effective delivery of the API. For a propellant to function satisfactorily in pMDIs, it needs to have a number of properties. These include an appropriate boiling point and vapor pressure so that it can be liquefied in a closed container at room temperature but develop a high enough pressure when the pMDI is activated to deliver the drug as an atomized formulation even at low ambient temperatures. Further, the propellant should be of low acute and chronic toxicity. It should have a high degree of chemical stability in contact with the drug, the container and the metallic and non-metallic components of the MDI device. The propellant preferably also is able to act as a carrier that is able to maintain the drug in a stable suspension or in a stable dispersion for a sufficient time after the suspension/dispersion is formed (e.g., by shaking for example) to permit reproducible delivery of the drug in use.

In a typical case, the user will shake the pMDI device in anticipation of use in order to establish the suspension. However, in some cases the length of time that the suspension is maintained without flocculation and/or aggregation can be undesirably short, which can potentially negatively impact the efficacy of delivering the active ingredient as intended. One factor which influences the tendency of flocculation and/or agglomeration to occur is the amount of the API in the suspension, with increasing amounts of API tending to aggravate the flocculation/agglomeration problem. Thus, it has been heretofore most common to overcome this problem by requiring the user to active the MDI more than once (usually twice) in order to introduce the desired dosage to the target area, thus permitting the amount of active ingredient contained in the carrier to be lower. For example, by requiring the user to activate the MDI twice to deliver the desired dosage, the amount of active ingredient suspended in the carrier can be reduced by 50%, thus reducing the flocculation/aggregation problem.

One object of the present invention is the development of a more environmentally friendly carrier for an API, especially fluticasone, which is able to avoid flocculation/aggregation problems, that is, that provides a pMDI formulation with excellent suspension stability. Achieving this result is a significant challenge because of the myriad of performance properties that at once must be achieved, especially with relatively high concentrations of active ingredients.

Carrier properties which can have an impact on pMDI performance can include appropriate boiling point and vapor pressure so that it can be liquefied in a closed container at room temperature but develop a high enough pressure when the pMDI is activated to deliver the drug as an atomized composition even at low ambient temperatures. Further, the carrier should be of low acute and chronic toxicity. It should have a high degree of chemical stability in contact with the drug, the container and the metallic and non-metallic components of the MDI device, and it should have a low propensity to extract low molecular weight substances from any elastomeric materials in the MDI device. The carrier preferably is able to maintain the drug, including relatively high concentrations of drug, in a stable suspension or in a stable dispersion for a sufficient time to permit reproducible delivery of the drug in use. When the drug is in suspension in the carrier, the density of the liquid carrier is desirably similar to that of the solid drug in order to avoid rapid sinking or floating of the drug particles in the liquid. Finally, the carrier should not present a significant flammability risk to the patient in use. In particular, it should form a non-flammable or low flammability mixture when mixed with air in the respiratory tract. Environmentally desirable properties, such as low GWP and low ODP, are also generally highly desirable.

U.S. Pat. No. 9,308,199, which is assigned to the assignee of the present application, describes the use of certain fluoroolefins, preferably hydrofluorolefins (HFO), as medicinally acceptable carriers that are able to overcome the environmental deficiencies of CFCs, HFCs and HCFCs mentioned above. Tetrafluoropropenes, including 1,3,3,3-tetrafluoropropene (HFO-1234ze) and 2,3,3,3-tetrafluoropropene (HFO-1234yf) are disclosed as being preferred.

WO2023/039103 mentions that HFOs have been proposed as propellants for MDIs, but it also notes that no MDI product has been successfully developed or commercialized using HFOs as a propellant. The '103 publication discloses an MDI that uses a formulation comprising greater than 70% by weight of HFO-1234ze(E), ethanol and at least one active pharmaceutical ingredient (API). The amounts of ethanol disclosed as being used in the '103 formulations range from as low as 0.1 wt. % to as high as 20%.

Asthma is one condition that has frequently been treated by the use of MDIs. Asthma has been described as a chronic disease that involves inflammation of the pulmonary airways and bronchial hyperresponsiveness that results in the clinical expression of a lower airway obstruction that usually is reversible. The pathophysiology of asthma or related disorders involves bronchoconstriction resulting from bronchial smooth muscle spasm and airway inflammation with mucosal edema. Treatment of asthma and other related disorders (such as seasonal allergies) has included the administration of the corticosteroid fluticasone. Two types of fluticasone are predominantly currently used, namely, fluticasone propionate (herein sometimes referred to for convenience as FP) and fluticasone furoate.

Notwithstanding the disclosures as mentioned above, applicants have come to recognize the need for delivery compositions, systems, devices and methods for fluticasone, including FP, that at once provide relatively low ozone depletion potential, relatively low global warming potential and the ability to maintaining the API, especially in relatively high concentrations, in a stable suspension or in a stable dispersion for a sufficient time to permit reproducible accurate delivery of the drug in use.

### SUMMARY

Applicants have found that many of the shortcomings of the prior compositions can be overcome and/or that many of the above-noted needs can be satisfied by pharmaceutical compositions of the present invention and the use thereof in MDIs and inhalation delivery methods.

The present invention includes pharmaceutical compositions comprising:
a. fine particles of fluticasone;
b. a carrier in which said fine particles of fluticasone are suspended, said carrier comprising: (i) from greater than 95% by weight to less than 99.5% by weight of HFO-1234ze(E); and (ii) from greater than 0.5% by weight to less than 5% by weight of ethanol.
   For the purposes of convenience, pharmaceutical compositions according to this paragraph are referred to as Pharmaceutical Composition 1A.

The present invention includes pharmaceutical compositions comprising:
a. fine particles of fluticasone;
b. a carrier in which said fine particles of fluticasone are suspended, said carrier comprising: (i) from greater than 97.5% by weight to less than 99.5% by weight of HFO-1234ze(E); and (ii) from greater than 0.5% by weight to less than 2.5% by weight of ethanol.
   For the purposes of convenience, pharmaceutical compositions according to this paragraph are referred to as Pharmaceutical Composition 1B.

The present invention includes pharmaceutical compositions comprising:
a. fine particles of fluticasone;
b. a carrier in which said fine particles of fluticasone are suspended, said carrier comprising: (i) from greater than 98% by weight to less than 99.5% by weight of HFO-1234ze(E); and (ii) from greater than 0.5% by weight to less than 2% by weight of ethanol.
   For the purposes of convenience, pharmaceutical compositions according to this paragraph are referred to as Pharmaceutical Composition 1C.

The present invention includes pharmaceutical compositions comprising:
a. fine particles of fluticasone;
b. a carrier in which said fine particles of fluticasone are suspended, said carrier comprising: (i) from greater than 98.5% by weight to less than 99.5% by weight of HFO-1234ze(E); and (ii) from greater than 0.5% by weight to less than 1.5% by weight of ethanol.
   For the purposes of convenience, pharmaceutical compositions according to this paragraph are referred to as Pharmaceutical Composition 1D.

The present invention includes pharmaceutical compositions comprising:
a. fine particles of fluticasone;
b. a carrier in which said fine particles of fluticasone are suspended, said carrier comprising: (i) about 99% by weight of HFO-1234ze(E); and (ii) about 1% by weight of ethanol.
   For the purposes of convenience, pharmaceutical compositions according to this paragraph are referred to as Pharmaceutical Composition 1E.

The present invention includes pharmaceutical compositions comprising:
a. fine particles of fluticasone;
b. a carrier in which said fine particles of fluticasone are suspended, said carrier comprising: (i) from greater than 95% by weight to less than 99.5% by weight of HFO-1234ze(E); and (ii) from greater than 0.5% by weight to less than 5% by weight of ethanol; and (iii) polyethylene glycol (“PEG).
   For the purposes of convenience, pharmaceutical compositions according to this paragraph are referred to as Pharmaceutical Composition 2A.

The present invention includes pharmaceutical compositions comprising:
a. fine particles of fluticasone propionate;
b. a carrier in which said fine particles of fluticasone propionate are suspended, said carrier comprising: (i) from greater than 95% by weight to less than 99.5% by weight of HFO-1234ze(E); (ii) from greater than 0.5% by weight to less than 5% by weight of ethanol; and (iii) a stability enhancing amount of polyethylene glycol (“PEG).
   For the purposes of convenience, pharmaceutical compositions according to this paragraph are referred to as Pharmaceutical Composition 2B.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) polyethylene glycol (“PEG).
   For the purposes of convenience, pharmaceutical compositions according to this paragraph are referred to as Pharmaceutical Composition 2C.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) polyethylene glycol (“PEG”) having a molecular weight of from about 400 to about 1000.
   For the purposes of convenience, pharmaceutical compositions according to this paragraph are referred to as Pharmaceutical Composition 2D.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) polyethylene glycol (“PEG”),
wherein said pharmaceutical composition has a TSI stability at 25 seconds of 25 or less.
   For the purposes of convenience, pharmaceutical compositions according to this paragraph are referred to as Pharmaceutical Composition 3A.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) polyethylene glycol (“PEG”),
wherein said pharmaceutical composition has a TSI stability at 25 seconds of 20 or less.
   For the purposes of convenience, pharmaceutical compositions according to this paragraph are referred to as Pharmaceutical Composition 3B.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) polyethylene glycol (“PEG”),
wherein said pharmaceutical composition has a TSI stability at 25 seconds of 16 or less.
   For the purposes of convenience, pharmaceutical compositions according to this paragraph are referred to as Pharmaceutical Composition 3C.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) from greater than 0.001% by weight to less than 0.01% by weight of polyethylene glycol (“PEG”).
   For the purposes of convenience, pharmaceutical compositions according to this paragraph are referred to as Pharmaceutical Composition 4A.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) from greater than 0.001% by weight to less than 0.0075% by weight of polyethylene glycol (“PEG”).
   For the purposes of convenience, pharmaceutical compositions according to this paragraph are referred to as Pharmaceutical Composition 4B.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) from greater than 0.0025% by weight to less than 0.0075% by weight of polyethylene glycol (“PEG”).
   For the purposes of convenience, pharmaceutical compositions according to this paragraph are referred to as Pharmaceutical Composition 4C.

The present invention includes pharmaceutical compositions comprising:
a. fine particles of fluticasone propionate;
b. a carrier in which said fine particles of fluticasone propionate are suspended, said carrier consisting essentially of: (i) from greater than 95% by weight to less than 99.5% by weight of HFO-1234ze(E); (ii) from greater than 0.5% by weight to less than 5% by weight of ethanol; and (iii) from greater than 0.004% by weight to less than 0.006% by weight of polyethylene glycol (“PEG”).
   For the purposes of convenience, pharmaceutical compositions according to this paragraph are referred to as Pharmaceutical Composition 4D.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.001% by weight to less than 0.01% by weight of polyethylene glycol (“PEG”).
   For the purposes of convenience, pharmaceutical compositions according to this paragraph are referred to as Pharmaceutical Composition 5A.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.001% by weight to less than 0.0075% by weight of polyethylene glycol (“PEG”).
   For the purposes of convenience, pharmaceutical compositions according to this paragraph are referred to as Pharmaceutical Composition 5B.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.0025% by weight to less than 0.0075% by weight of polyethylene glycol (“PEG”).
   For the purposes of convenience, pharmaceutical compositions according to this paragraph are referred to as Pharmaceutical Composition 5C.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.004% by weight to less than 0.006% by weight of polyethylene glycol (“PEG”).
   For the purposes of convenience, pharmaceutical compositions according to this paragraph are referred to as Pharmaceutical Composition 5D.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.001% by weight to less than 0.01% by weight of polyethylene glycol (“PEG”) having an average molecular weight of from about 300 to about 1200.
   For the purposes of convenience, pharmaceutical compositions according to this paragraph are referred to as Pharmaceutical Composition 6A.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.001% by weight to less than 0.0075% by weight of polyethylene glycol (“PEG”) having an average molecular weight of from about 300 to about 1200.
   For the purposes of convenience, pharmaceutical compositions according to this paragraph are referred to as Pharmaceutical Composition 6B.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.0025% by weight to less than 0.0075% by weight of polyethylene glycol (“PEG”) having an average molecular weight of from about 300 to about 1200.
   For the purposes of convenience, pharmaceutical compositions according to this paragraph are referred to as Pharmaceutical Composition 6C.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.004% by weight to less than 0.006% by weight of polyethylene glycol (“PEG”) having an average molecular weight of from about 300 to about 1200.
   For the purposes of convenience, pharmaceutical compositions according to this paragraph are referred to as Pharmaceutical Composition 6D.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.001% by weight to less than 0.01% by weight of polyethylene glycol (“PEG”) having an average molecular weight of from about 800 to about 1200.
   For the purposes of convenience, pharmaceutical compositions according to this paragraph are referred to as Pharmaceutical Composition 7A.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.001% by weight to less than 0.0075% by weight of polyethylene glycol (“PEG”) having an average molecular weight of from about 800 to about 1200.
   For the purposes of convenience, pharmaceutical compositions according to this paragraph are referred to as Pharmaceutical Composition 7B.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.0025% by weight to less than 0.0075% by weight of polyethylene glycol (“PEG”) having an average molecular weight of from about 800 to about 1200.
   For the purposes of convenience, pharmaceutical compositions according to this paragraph are referred to as Pharmaceutical Composition 7C.

The present invention includes pharmaceutical compositions comprising:
a. fine particles of fluticasone propionate;
b. a carrier in which said fine particles of fluticasone propionate are suspended, said carrier consisting essentially of: (i) from greater than 97.5% by weight to less than 99.5% by weight of HFO-1234ze(E); (ii) from greater than 0.5% by weight to less than 2.5% by weight of ethanol; and (iii) from greater than 0.004% by weight to less than 0.006% by weight of polyethylene glycol (“PEG”) having an average molecular weight of from about 800 to about 1200.
   For the purposes of convenience, pharmaceutical compositions according to this paragraph are referred to as Pharmaceutical Composition 7D.

The present invention includes pharmaceutical compositions comprising:
a. fine particles of fluticasone propionate;
b. a carrier in which said fine particles of fluticasone propionate are suspended, said carrier consisting essentially of: (i) from greater than 98.5% by weight to less than 99.5% by weight of HFO-1234ze(E); (ii) from greater than 0.5% by weight to less than 1.5% by weight of ethanol; and (iii) from greater than 0.003% by weight to less than 0.008% by weight of polyethylene glycol (“PEG”) having an average molecular weight of from about 800 to about 1200.
   For the purposes of convenience, pharmaceutical compositions according to this paragraph are referred to as Pharmaceutical Composition 8A.

The present invention includes pharmaceutical compositions comprising:
a. fine particles of fluticasone propionate;
b. a carrier in which said fine particles of fluticasone propionate are suspended, said carrier consisting essentially of: (i) from greater than 98.5% by weight to less than 99.5% by weight of HFO-1234ze(E); (ii) from greater than 0.5% by weight to less than 1.5% by weight of ethanol; and (iii) from greater than 0.001% by weight to less than 0.01% by weight of polyethylene glycol (“PEG”) having an average molecular weight of from about 800 to about 1200.
   For the purposes of convenience, pharmaceutical compositions according to this paragraph are referred to as Pharmaceutical Composition 8B.

The present invention includes pharmaceutical compositions comprising:
a. fine particles of fluticasone propionate;
b. a carrier in which said fine particles of fluticasone propionate are suspended, said carrier consisting essentially of: (i) from greater than 98.5% by weight to less than 99.5% by weight of HFO-1234ze(E); (ii) from greater than 0.5% by weight to less than 1.5% by weight of ethanol; and (iii) from greater than 0.0025% by weight to less than 0.0075% by weight of polyethylene glycol (“PEG”) having an average molecular weight of from about 800 to about 1200.
   For the purposes of convenience, pharmaceutical compositions according to this paragraph are referred to as Pharmaceutical Composition 8C.

The present invention includes pharmaceutical compositions comprising:
a. fine particles of fluticasone propionate;
b. a carrier in which said fine particles of fluticasone propionate are suspended, said carrier consisting essentially of: (i) from greater than 98.5% by weight to less than 99.5% by weight of HFO-1234ze(E); (ii) from greater than 0.5% by weight to less than 1.5% by weight of ethanol; and (iii) from greater than 0.004% by weight to less than 0.006% by weight of polyethylene glycol (“PEG”) having an average molecular weight of from about 800 to about 1200.
   For the purposes of convenience, pharmaceutical compositions according to this paragraph are referred to as Pharmaceutical Composition 8D.

The present invention also includes methods for delivering of fluticasone comprising:
a. providing in an MDI comprising: (1) a carrier comprising from greater than about 95% by weight to less than 99.5% by weight of HFO-1234ze(E); (ii) from greater than 0.5% by weight to less than about 5% by weight of ethanol; and (2) fine particles of fluticasone propionate; and
b. actuating said MDI to produce an aerosol spray of said fluticasone, wherein said spray has a TSI stability at 30 seconds of 25 or less.
   For the purposes of convenience, methods according to this paragraph are referred to as Pharmaceutical Delivery Method 1A.

The present invention also includes methods for delivering of fluticasone propionate comprising:
a. providing in an MDI comprising: (1) a carrier comprising from greater than about 95% by weight to less than 99.5% by weight of HFO-1234ze(E); (ii) from greater than 0.5% by weight to less than about 5% by weight of ethanol; and (2) fine particles of fluticasone propionate; and
b. actuating said MDI to produce an aerosol spray of said fluticasone propionate, wherein said spray has a TSI stability at 30 seconds of 20 or less.
   For the purposes of convenience, methods according to this paragraph are referred to as Pharmaceutical Delivery Method 1B.

The present invention also includes methods for delivering a dose of fluticasone comprising:
a. providing an MDI which comprises a pharmaceutical composition of the present invention, including each of Pharmaceutical Composition 1-10; and
b. actuating said MDI to produce an aerosol spray of fluticasone fine particles.
   For the purposes of convenience, methods according to this paragraph are referred to as Pharmaceutical Delivery Method 3A.

The present invention also includes methods for delivering a prescribed dose of fluticasone comprising:
a. providing in an MDI which comprises a pharmaceutical composition of the present invention, including each of Pharmaceutical Composition 1-10; and
b. actuating said MDI once to produce a single aerosol spray of fluticasone fine particles, wherein said single spray contains about the prescribed dose of said fluticasone.
   For the purposes of convenience, methods according to this paragraph are referred to as Pharmaceutical Delivery Method 3B.

The present invention also includes methods for delivering a prescribed dose of fluticasone comprising:
a. providing in an MDI which comprises a pharmaceutical composition of the present invention, including each of Pharmaceutical Composition 1-10; and
b. actuating said MDI once to produce a single aerosol spray of fluticasone fine particles, wherein said single spray contains about the prescribed dose of said fluticasone, wherein said spray wherein said spray has a TSI stability at 30 seconds of 25 or less.
   For the purposes of convenience, methods according to this paragraph are referred to as Pharmaceutical Delivery Method 3C.

The present invention also includes methods for delivering a prescribed dose of fluticasone comprising:
a. providing in an MDI which comprises a pharmaceutical composition of the present invention, including each of Pharmaceutical Composition 1-10; and
b. actuating said MDI once to produce a single aerosol spray of fluticasone fine particles, wherein said single spray contains about the prescribed dose of said fluticasone, wherein said spray wherein said spray has a TSI stability at 30 seconds of 20 or less.
   For the purposes of convenience, methods according to this paragraph are referred to as Pharmaceutical Delivery Method 3D.

The present invention also includes methods for delivering a prescribed dose of fluticasone comprising:
a. providing in an MDI which comprises a pharmaceutical composition of the present invention, including each of Pharmaceutical Composition 1-10; and
b. actuating said MDI once to produce a single aerosol spray of fluticasone fine particles, wherein said single spray contains about the prescribed dose of said fluticasone, wherein said spray wherein said spray has a TSI stability at 30 seconds of 20 or less.
   For the purposes of convenience, methods according to this paragraph are referred to as Pharmaceutical Delivery Method 3E.

The present invention also includes methods for delivering a prescribed dose of fluticasone propionate comprising:
a. providing in an MDI comprising: (1) a carrier comprising from greater than about 95% by weight to less than 99% by weight of HFO-1234ze(E); and (ii) from greater than 1% by weight to less than about 5% by weight of ethanol; and (2) fine particles of the API carried by said carrier, wherein said API comprises fluticasone propionate; and
b. actuating said MDI once to produce a single aerosol spray of said pharmaceutical composition, wherein said single spray contains said prescribed dose, wherein said prescribed dose is from about 150 micrograms to about 300 micrograms.
   For the purposes of convenience, methods according to this paragraph are referred to as Pharmaceutical Delivery Method 4.

The present invention also includes methods for delivering a prescribed dose of fluticasone comprising:
a. providing in an MDI which delivers an aerosol spray of a pharmaceutical composition of the present invention, including each of Pharmaceutical Composition 1-10; and
b. actuating said MDI once to produce an aerosol spray of said pharmaceutical composition, wherein said spray contains said prescribed dose, wherein said prescribed dose is an amount of from about 150micrograms to about 300 micrograms of fluticasone.
   For the purposes of convenience, methods according to this paragraph are referred to as Pharmaceutical Delivery Method 5.

The present invention also includes a metered dose inhaler (MDI) comprising:
a. a container;
b. a pharmaceutical composition under pressure in the container, said pharmaceutical composition comprising: (i) from greater than about 95% by weight to less than 99% by weight of HFO-1234ze(E); (ii) from greater than 1% by weight to less than about 5% by weight of ethanol; (iii) from greater than 0.001% by weight to less than about 0.05% by weight of polyethylene glycol (“PEG”); and (iv) from about 0.1% by weight to about 0.5% by weight of fluticasone propionate, said fluticasone propionate being in the form of fine particles; and
c. a normally closed valve on the container, said normally closed valve being actuatable to an open position to release an aerosol spray of said pharmaceutical composition from said container.
   For the purposes of convenience, MDIs according to this paragraph are referred to as MDI 1A.

The present invention also includes a metered dose inhaler (MDI) comprising:
a. a container;
b. a pharmaceutical composition of the present invention, including each of Pharmaceutical Composition 1-10, under pressure in the container; and
c. a normally closed valve on the container, said normally closed valve being actuatable to an open position to release an aerosol spray of said pharmaceutical composition from said container.
   For the purposes of convenience, MDIs according to this paragraph are referred to as MDI 1B.

The present invention also includes a metered dose inhaler (MDI) comprising:
a. a container;
b. a pharmaceutical composition under pressure in said container, said pharmaceutical composition comprising: (a) a carrier comprising from greater than about 95% by weight to less than 99% by weight of HFO-1234ze(E); and (ii) from greater than 1% by weight to less than about 5% by weight of ethanol; and (b) from about 1 mg/mL to about 6 mg/ml of fine particles of fluticasone propionate suspended in said carrier
   For the purposes of convenience, MDIs according to this paragraph are referred to as MDI 2A.

The present invention also includes a metered dose inhaler (MDI) comprising:
a. a container;
b. a pharmaceutical composition under pressure in said container, said pharmaceutical composition comprising: (a) a carrier comprising from greater than about 95% by weight to less than 99% by weight of HFO-1234ze(E); and (ii) from greater than 1% by weight to less than about 5% by weight of ethanol; and (b) from about 1 mg/mL to about 5 mg/mL of fine particles of fluticasone propionate suspended in said carrier
   For the purposes of convenience, MDIs according to this paragraph are referred to as MDI 2B.

The present invention also includes a metered dose inhaler (MDI) comprising:
a. a container;
b. a pharmaceutical composition under pressure in said container, said pharmaceutical composition being at least one of Pharmaceutical Compositions 1-10 and comprising from about 1 mg/mL to about 3 mg/mL of fine particles of fluticasone propionate.
   For the purposes of convenience, MDIs according to this paragraph are referred to as MDI 3A.

The present invention also includes a metered dose inhaler (MDI) comprising:
a. a container;
b. a pharmaceutical composition under pressure in said container, said pharmaceutical composition being at least one of Pharmaceutical Compositions 1-10 and comprising from about 3 mg/mL to about 5 mg/mL of fine particles of fluticasone propionate.
   For the purposes of convenience, MDIs according to this paragraph are referred to as MDI 3B.

The present invention also includes a metered dose inhaler (MDI) comprising:
a. a container;
b. a pharmaceutical composition under pressure in said container, said pharmaceutical composition being at least one of Pharmaceutical Compositions 1-10, wherein said MDI produces a spray with an RSD % of about 15% or less.
   For the purposes of convenience, MDIs according to this paragraph are referred to as MDI 3C.

### BRIEF DESCRIPTION OF THE DRAWING

The invention will now be described with reference to the accompanying drawings in which:

FIG. 1 is a cross-sectional side view of an inhaler including a canister containing a valve according to the present disclosure.

FIG. 2 is a detailed cross-sectional side view of the inhaler of FIG. 1.

FIG. 3 is a cross-sectional side view of a metering valve for an inhaler.

FIG. 4 is a chart illustrating the results of Comparative Example 1.

FIG. 5 is a chart illustrating the results of Comparative Examples 3A-3C.

FIG. 6 is a chart illustrating the results of Examples 1A-1B.

FIG. 7 is a chart illustrating the results of Examples 2A-2C.

FIG. 8 is a chart illustrating the results of Example 3.

FIG. 9 is a chart illustrating the results of Example 4.

### DETAILED DESCRIPTION OF PREFERRED EMBODIMENTS

### I. Definitions

For the purposes of this invention, the term “about” in relation to the amounts expressed in weight percent for amounts greater than 2% means that the amount of the component can vary by an amount of +/−10% relative by weight. For example, the amount of “about 2%” means 2%+/−0.2%.

For the purposes of this invention, the term “about” in relation to the amounts expressed in milligrams/milliliter (mg/mL) means that the amount of the component can vary by an amount of +/−0.05 mg/mL.

For the purposes of this invention, the term “stability enhancing amount” means an amount of the indicated component to produce at least a 20% relative improvement in TSI stability measured at 30 seconds.

For the purposes of this invention, the term “about” in relation to the amounts of a prescribed dose expressed in micrograms (μg) means that the amount of the component can vary by an amount of +/−5 μg.

For the purposes of this invention, the term “composition” is used in the broad sense to include both single phase compositions and compositions that comprise two or more phases, such as for example compositions comprising a continuous liquid phase and solid particles suspended or dispersed in the liquid phase, or for example a gaseous phase with solid particles suspended, dispersed and/or carried by the gaseous phase, as would occur in an aerosol composition.

The term “pharmaceutical composition” is used herein to include any composition which comprises at least one agent, ingredient, drug, compound, composition, or other substance that may be used on, or administered to, a human or animal for a purpose that includes one or more of therapeutic, pharmaceutical, pharmacological, diagnostic, and prophylactic and immunomodulation.

The term “fine particles” means particles that have of a mean particle diameter up to about 10 microns (10μ).

The term “prescribed dose” refers to the amount of fluticasone, including fluticasone propionate, intended by the manufacturer of an MDI or by a medical professional to be taken at one specified time.

The term “RSD %” means the relative standard deviation of a delivered dose as measured in accordance with the examples hereof.

As used herein, the term “carrier” refers to one or more pharmacologically inert substances which provide a continuous phase in which fluticasone particles (including fluticasone propionate particles) are suspended and which comprise components that exert a sufficiently high vapor pressure at normal room temperature to propel the particles from the canister of an MDI to a patient upon actuation of the MDI's metering valve. Therefore, the term “carrier” encompasses both a single component and a combination of two or more different components that form the medium in which the fluticasone is suspended or otherwise carried. Thus, the 1234ze(E) component of the carrier of present composition acts at least as a propellent.

The term “respirable” generally refers to particles, aggregates, drops, etc. sized such that they can be inhaled and reach the airways of the lung.

The term “HFC-134a” means 1,1,1,2-tetrafluoroethane.

The terms “HFO-1234ze(E),” and “1234ze(E)” as used herein each mean trans-1,3,3,3-tetrafluoropropene. Unless otherwise stated, “HFO-1234ze” and “1234ze” mean trans-1,3,3,3-tetrafluoropropene.

The term “fluticasone” as used herein encompasses any and all pharmaceutically acceptable versions of fluticasone, including pharmaceutically acceptable esters of fluticasone (such as fluticasone propionate and fluticasone furoate). Commercially available formulations containing fluticasone propionate are available under the trade names Flovent, Flixotide, Flonase and others.

Reference herein to a group of defined items includes all such defined items, including all such items with suffix designations. Thus for example, a reference herein to “Pharmaceutical Compositions 1-10,” is a specific reference to each of Pharmaceutical Compositions 1A, 1B, 1C, 1D, 2A, 2B, 2C, 2D, 2E, 2F, 2G and so on.

### II. The Compositions

The preferred pharmaceutical compositions of the present invention, including each of Pharmaceutical Compositions 1-10, are preferably suspensions of the fluticasone propionate in the carrier comprising the other required components of the composition, including particularly the HFO-1234ze(E) and ethanol.

The preferred pharmaceutical compositions of the present invention, including each of Pharmaceutical Compositions 1-10, are physically stable. The preferred pharmaceutical compositions of the present invention, including each of Pharmaceutical Compositions 1-10, are chemically stable. The preferred pharmaceutical compositions of the present invention, including each of Pharmaceutical Compositions 1-10, are physically stable and chemically stable.

### Concentration of Components

The concentration of the components in the present compositions can generally vary widely within the broad scope of the present invention. The concentration of the fluticasone contained in the compositions of the present invention, including each of Pharmaceutical Compositions 1-10, measured as milligrams per milliliter (mg/mL) is preferably from greater than 2.5 mg/mL, or from greater than about 2.5 mg/mL to less than about 5 mg/mL, or from greater than about 2.5 mg/mL to less than about 4.5 mg/mL, or from greater than 2.5 mg/mL to less than about 4.0 mg/mL. Preferred compositions include those identified in the following Tables 1A and 1B below, with the following designations in the table having the following meanings: “Comp” means that the composition comprises the identified components; CEO means that the composition consists essentially of the identified components; CO means that the composition consists of the identified components; and FP means fluticasone propionate; and NR means that the component or property is not required (but may be present) according to the definition.

TABLE 1A

| Pharma- | | | | | | TSI |
|---|---|---|---|---|---|---|
| ceutical | | | | | | stability |
| Composition | | Components, wt. % | | | | at 30 |
| No. | | 1234ze(E) | Ethanol | PEG | FP | seconds |
| PC9A | Comp | =>97.5-=<99.5 | =>0.5-=<2.5 | NR | 0.1-<0.5 | NR |
| PC9B | CEO | =>98-=<99.5 | =>0.5-=<2 | NR | 0.1-<0.5 | NR |
| PC9C | CO | =>98.5-=<99.5 | =>0.5-=<1.5 | NR | 0.1-<0.5 | NR |
| PC9D | Comp | about 99 | about 1 | NR | 0.1-<0.5 | NR |
| PC9E | Comp | =>97.5-=<99.5 | =>0.5-=<2.5 | NR | 0.1-<0.5 | =<25 |
| PC9F | CEO | =>98-=<99.5 | =>0.5-=<2 | NR | 0.1-<0.5 | =<25 |
| PC9G | CO | =>98.5-=<99.5 | =>0.5-=<1.5 | NR | 0.1-<0.5 | =<25 |
| PC9H | Comp | about 99 | about 1 | NR | 0.1-<0.5 | =<25 |
| PC9I | Comp | =>97.5-=<99.5 | =>0.5-=<2.5 | =>0.001-=<0.01 | 0.1-<0.5 | =<20 |
| PC9J | CEO | =>98-=<99.5 | =>0.5-=<2 | =>0.001-=<0.01 | 0.1-<0.5 | =<20 |
| PC9K | CO | =>98.5-=<99.5 | =>0.5-=<1.5 | =>0.001-=<0.01 | 0.1-<0.5 | =<20 |
| PC9L | Comp | about 99 | about 1 | =>0.001-=<0.01 | 0.1-<0.5 | =<20 |
| PC9M | Comp | =>97.5-=<99.5 | =>0.5-=<2.5 | =>0.001-=<0.01 | 0.1-<0.5 | =<17 |
| PC9N | CEO | =>98-=<99.5 | =>0.5-=<2 | =>0.001-=<0.01 | 0.1-<0.5 | =<17 |
| PC90O | CO | =>98.5-=<99.5 | =>0.5-=<1.5 | =>0.001-=<0.01 | 0.1-<0.5 | =<17 |
| PC9P | Comp | about 99 | about 1 | =>0.001-=<0.01 | 0.1-<0.5 | =<17 |
| PC10A | Comp | about 99 | about 1 | NR | 0.1-<0.5 | =<25 |
| PC10B | Comp | about 99 | about 1 | about 0.005 | 0.1-<0.5 | =<25 |
| PC10C | CEO | about 99 | about 1 | about 0.005 | 0.1-<0.5 | =<25 |
| PC10D | CO | about 99 | about 1 | about 0.005 | 0.1-<0.5 | =<25 |
| PC10E | Comp | about 99 | about 1 | about 0.005 | 0.1-<0.5 | =<20 |
| PC10F | CEO | about 99 | about 1 | about 0.005 | 0.1-<0.5 | =<20 |
| PC10G | CO | about 99 | about 1 | about 0.005 | 0.1-<0.5 | =<20 |
| PC10H | Comp | about 99 | about 1 | about 0.005 | 0.1-<0.5 | =<18 |
| PC10I | CEO | about 99 | about 1 | about 0.005 | 0.1-<0.5 | =<18 |
| PC10J | CO | about 99 | about 1 | about 0.005 | 0.1-<0.5 | =<18 |
| PC10K | Comp | about 99 | about 1 | about 0.005 | 0.1-<0.5 | =<17 |
| PC10L | CEO | about 99 | about 1 | about 0.005 | 0.1-<0.5 | =<17 |
| PC10M | CO | about 99 | about 1 | about 0.005 | 0.1-<0.5 | =<17 |
| PC10N | Comp | about 99 | about 1 | about 0.005 | 0.1-<0.5 | =<16 |
| PC10O | CEO | about 99 | about 1 | about 0.005 | 0.1-<0.5 | =<16 |
| PC10P | CO | about 99 | about 1 | about 0.005 | 0.1-<0.5 | =<16 |

For all compositions of the present invention, other than those defined as “consisting of” the designated components, including each of Pharmaceutical Compositions 1-10, additional components or excipients may be present. These components may have various uses and functions, including, but not limited to, facilitating formation of a suspension, stabilizing a suspension, and/or aiding in chemical stabilization of fluticasone or other components.

Preferred excipients include are suitable for inhaled delivery and do not substantially degrade or dissolve in the suspension medium, and preferably are suspension formation aids, suspension stabilizers, fluticasone stabilizers and combinations of these. In particular cases, the excipient is selected from the group consisting of lipids, phospholipids, carbohydrates, amino acids, organic salts, peptides, proteins, alditols, synthetic or natural polymers, surfactant materials and combinations of these.

For all compositions of the present invention, including each of Pharmaceutical Compositions 1-10, the composition comprises a suspension of the indicated fluticasone in the HFO-1234ze(E) and ethanol, and preferably PEG when present. In all such compositions, including each of Pharmaceutical Compositions 1-10, the designated fluticasone is preferably in a microparticulate solid form (preferably micronized, but it can also be size-reduced by a multitude of other particle size reduction techniques). As used herein, a suspension of fluticasone may also have a very small amount of solubilized particulate material within the composition. For the present compositions, including each of Pharmaceutical Compositions 1-10, solubilization of fluticasone is generally undesirable. In embodiments, including each of Pharmaceutical Compositions 1-10, there may exists a minimal or nominal solubilization of the fluticasone, but in preferred embodiments including each of Pharmaceutical Compositions 1-10, there is essentially no measurable solubilization of the fluticasone.

In certain preferred forms, pMDIs of the present invention, including those pMDIs containing a compositions of the present invention, including each of Pharmaceutical Compositions 1-10, have an RSD % of less than about 15%.

In certain preferred forms, the compositions of the present invention, including each of Pharmaceutical Compositions 1-10, have a Global Warming Potential (GWP) of not greater than about 300, more preferably not greater than about 150, not greater than 75, and most preferably not greater than about 10. As used herein, “GWP” is measured relative to that of carbon dioxide and over a 100-year time horizon, as defined in “The Scientific Assessment of Ozone Depletion, 2002, a report of the World Meteorological Association's Global Ozone Research and Monitoring Project,” which is incorporated herein by reference.

In certain preferred forms, the present compositions also preferably have an Ozone Depletion Potential (ODP) of not greater than 0.05, more preferably not greater than 0.02 and even more preferably about zero. As used herein, “ODP” is as defined in “The Scientific Assessment of Ozone Depletion, 2002, A report of the World Meteorological Association's Global Ozone Research and Monitoring Project,” which is incorporated herein by reference.

### III. Devices and Methods

The present invention includes devices for the delivery by inhalation the composition of the present invention, including each of Pharmaceutical Compositions 1-10. In certain preferred embodiments, the devices of the present invention comprise a container, preferably an aerosol canister, containing a pressurized composition of the present invention, including each of Pharmaceutical Compositions 1-10, and preferably having a metered dose dispensing valve operable between non-dispensing and dispensing positions. The present devices preferably also comprise an actuator, which in preferred embodiments comprises a housing adapted to receive the aerosol container and to define a chamber in fluid communication with a patient port for introducing the medicament into the oral and/or nasal cavity of the patient, preferably in the form of a mouthpiece and/or nasal adapter. The actuator also preferably includes a nozzle block adapted to receive the valve stem of the dispensing valve, the nozzle block preferably comprising a passage in fluid communication with the valve stem and terminating in an orifice for directing medicament from the valve stem into the chamber.

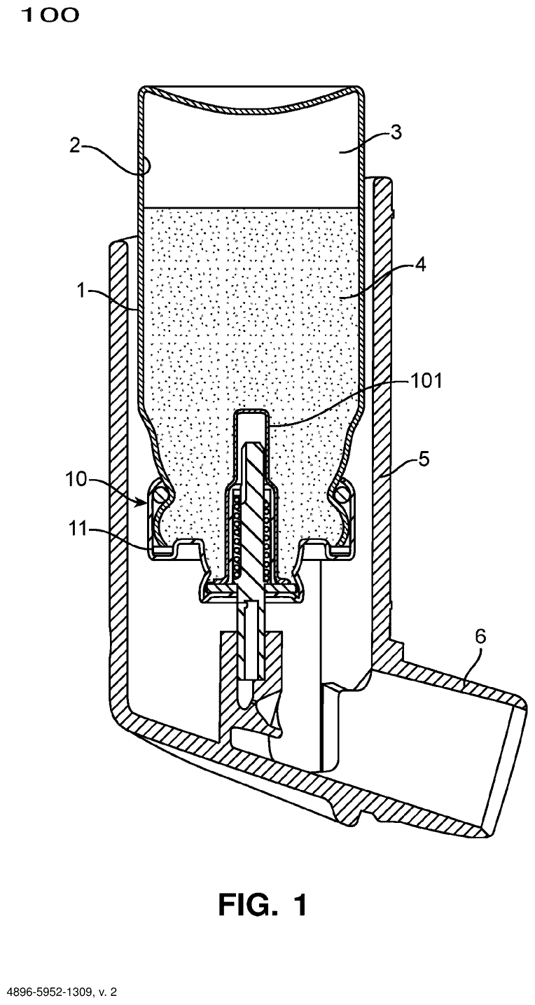

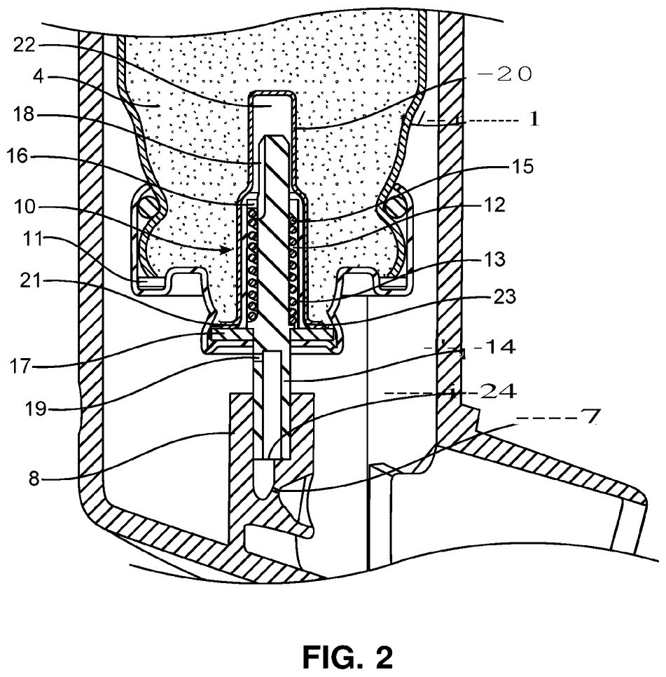

By way of example but not by way of limitation, FIG. 1 shows one embodiment of a metered dose inhaler 100, including an aerosol canister 1 fitted with a metered dose metering valve 10 (shown in its resting position). The metering valve 10 is typically affixed, i.e., crimped, onto the canister via a cap or ferrule 11 (typically made of aluminum or an aluminum alloy) which is generally provided as part of the valve assembly. Between the canister and the ferrule there may be one or more seals. In the embodiments shown in FIG. 1 and FIG. 2 between the canister 1 and the ferrule 11 there are two seals including, e.g., an O-ring seal and a gasket seal.

As shown in FIG. 1, the canister/valve dispenser is typically provided with an actuator 5 including an appropriate patient port 6, such as a mouthpiece. For administration to the nasal cavities the patient port is generally provided in an appropriate form (e.g., smaller diameter tube, often sloping upwardly) for delivery through the nose. Actuators are generally made of a plastic material, for example polypropylene or polyethylene. As can be seen from FIG. 1, inner walls 2 of the canister and outer walls 101 of the portion(s) of the metering valve 10 located within the canister define a formulation chamber 3 in which aerosol composition 4 is contained.

The valve 10 shown in FIG. 1 and FIG. 2, includes a metering chamber 12, defined in part by an inner valve body 13, through which a valve stem 14 passes. The valve stem 14, which is biased outwardly by a compression spring 15, is in sliding sealing engagement with an inner tank seal 16 and an outer diaphragm seal 17. The valve 10 also includes a second valve body 20 in the form of a bottle emptier. The inner valve body 13 (also referred to as the “primary” valve body) defines in part the metering chamber 12. The second valve body 20 (also referred to as the “secondary” valve body) defines in part a pre-metering region or chamber besides serving as a bottle emptier.

Referring to FIG. 2, aerosol composition 4 can pass from the composition chamber 3 into a pre-metering chamber 22 provided between the secondary valve body 20 and the primary valve body 13 through an annular space 21 between a flange 23 of the secondary valve body 20 and the primary valve body 13. To actuate (fire) the valve 10, the valve stem 14 is pushed inwardly relative to the canister 1 from its resting position shown in FIG. 1 and FIG. 2, allowing composition to pass from the metering chamber 12 through a side hole 19 in the valve stem and through a stem outlet 24 to an actuator nozzle 7 then out to the patient. When the valve stem 14 is released, composition enters into the valve 10, in particular into the pre-metering chamber 22, through the annular space 21 and thence from the pre-metering chamber through a groove 18 in the valve stem past the tank seal 16 into the metering chamber 12.

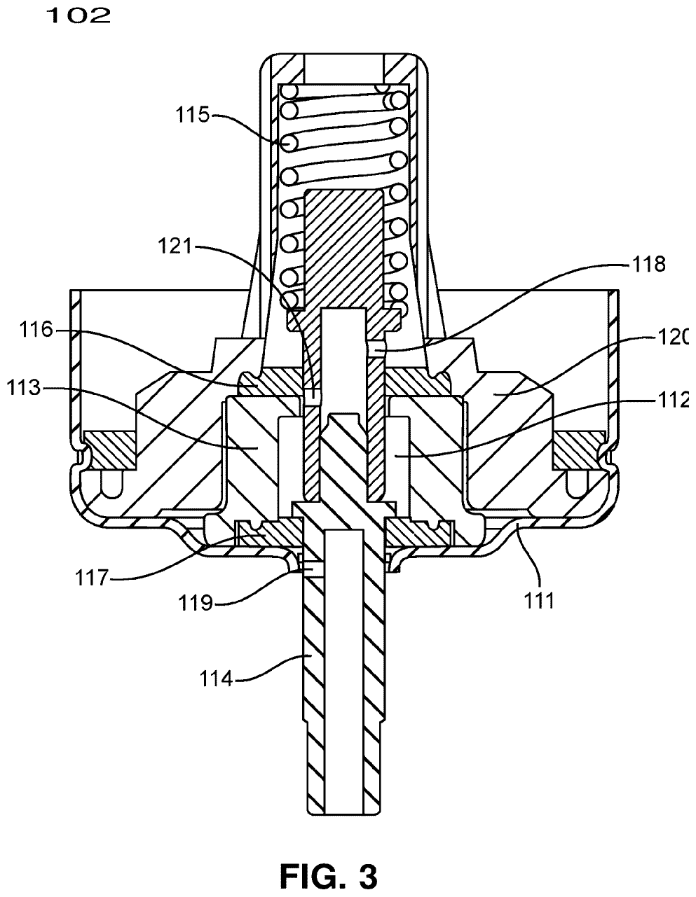

FIG. 3 shows another embodiment of a metered dose aerosol metering valve 102, different from the embodiment shown in FIG. 1 and FIG. 2, in its rest position. The valve 102 has a metering chamber 112 defined in part by a metering tank 113 through which a stem 114 is biased outwardly by spring 115. The stem 114 is made in two parts that are push fit together before being assembled into the valve 102. The stem 114 has an inner seal 116 and an outer seal 117 disposed about it and forming sealing contact with the metering tank 113. A valve body 120 crimped into a ferrule 111 retains the aforementioned components in the valve. In use, composition enters the metering chamber via orifices 121 and 118. The composition's outward path from the metering chamber 112 when a dose is dispensed is via orifice 119.

In certain embodiments the invention device is constructed such that airflow due to patient inhalation is prevented or reduced in the vicinity of the orifice at all times or only during dispensing of the medicament from the valve. Either of such arrangements has the effect of substantially reducing the velocity of the emitted spray compared to an inhaler which allows free flow of air in the vicinity of the nozzle block during dispensing of the medicament.

In certain embodiments, the actuator is constructed such that the distance from the nozzle to the mouthpiece is from approximately 1 to 15 cm, preferably 4 to 6 cm, with a chamber/mouthpiece diameter from 1 to 4 cm, 0.5 to 1 cm in the case of a nasal adapter.

In certain preferred but non-limiting embodiments, the actuator possess air inlets which enable the patient to inhale though the patient port, preferably without encountering significant resistance since the patient may have breathing difficulties when taking the medication, for example, during an asthma attack. However, the air inlets, for example in the mouthpiece, preferably do not concentrate the airflow into an area that is too narrow, as this will give a high velocity of incoming air which will deflect the spray onto the wall of the mouthpiece opposite the air inlets. In certain preferred embodiments the air inlets are positioned downstream of the nozzle, in the region of the turbulent zone and/or downstream of the turbulent zone. The positioning and direction of the air inlets may also affect the deposition of medicament within the chamber and mouthpiece. In one arrangement air inlets comprise a series of holes and optionally may be interdispersed with fluid deflection structures on the wall of the chamber, to direct air into the turbulent zone to mix air with the aerosol stream. Further, the mouthpiece may be constructed of porous material to allow a multiplicity of finely divided air vents to provide air flow over a larger surface area.

In certain embodiments the actuator possesses air inlets upstream of or in the vicinity of the nozzle, but the air inlets are blocked when the valve is fired to release the aerosol spray. The air inlets are opened after the spray has been released by which time the velocity of the stream will have been reduced and the turbulent zone formed. Upon inhalation, an airflow is established from the air inlets to the mouthpiece which entrains the residual aerosol spray. The actuator may include additional air inlets downstream of the nozzle, as described above with respect to the first embodiment. These downstream air inlets do not need to close during release of the aerosol spray.

In certain embodiments, a porous membrane is present to introduce air into or downstream of the turbulent zone. One advantage of the use of such a membrane is that the air is introduced more uniformly and diffusely around the circumference of the spray, thereby acting as a buffer between the turbulent flow and the wall. The effect is to reduce drug deposition in the device. The membrane may optionally be protected from dirt or contact by the user's lips by an additional part of the mouthpiece. When present, it is preferred that the porous membrane material (50) must not significantly impede the patient's ability to inhale through the device. A suitable material is Whatmann No. 4 filter paper; but other materials may be used, such as those used in cylindrical air filters or membrane filters, or such as those formed by sintering polymers. A preferred porous membrane material is in the form of a cylinder made by fusing together small pellets of polypropylene.

For certain medicaments, it is preferred to configure the device so as to reduce contact between the medicament and parts of the patient's body that it is not intended to contact. For example, residues of the medicament deposited on internal surfaces of actuators may be fingered and transferred to other body parts. In such cases, the device may be configured to include one or more fluid flow deflectors to allow the spray to pass through, whilst limiting access by the patient to internal surfaces of the actuator. Of course, the device may be configured for intranasal delivery. This is normally quite undesirable, since the medicaments were designed for delivery to the respiratory system and may not have an appropriate effect when deposited in the oropharynx and allowed to enter the digestive tract. In an effort to overcome this problem, certain embodiments of the present device include the provision of a holding volume, commonly called a spacer, in which the medicament is fired. The spacer preferably allows the velocity of the medicament to be reduced and may also allow some propellant evaporation to occur. Spacers can improve the performance of a metered dose inhaler by reducing oropharyngeal deposition.

The total amount of composition of the present invention, including each of Pharmaceutical Compositions 1-10, contained in the canister preferably is selected so that at least a portion of the propellant in the canister is present as a liquid after a predetermined number of medicinal doses have been delivered. The predetermined number of doses may be 5 to 200, 30 to 200, 60 to 200, 60 to 120, 60, 120, 200, or any other number of doses. In preferred embodiments, the total amount of composition of the present invention, including each of Pharmaceutical Compositions 1-10, in the canister may be from 1.0 grams (g) to 30.0 g, 2.0 g to 20.0 g, or 5.0 to 10.0 g. The total amount of composition of the present invention, including each of Pharmaceutical Compositions 1-10, is typically selected to be greater than the product of the predetermined number of doses and the metering volume of the metering valve. In some embodiments, the total amount of composition is greater than 1.1 times, greater than 1.2 times, greater than 1.3 times, greater than 1.4 times, or greater than 1.5 times the product of the predetermined number of doses and the metering volume of the metering valve. This helps to ensure that the amount of each dose remains relatively constant through the life of the inhaler.

The present invention thus provides inhalers, and preferably metered dose inhalers (MDIs) for the treatment of asthma and other chronic obstructive pulmonary diseases and for delivery of pharmaceutical compositions, including each of Pharmaceutical Compositions 1-10, to the lungs, preferably intraorally and/or to accessible mucous membranes or intranasally. The present invention thus includes methods for delivering of pharmaceutical compositions, including each of Pharmaceutical Compositions 1-10, for purpose of treating ailments, diseases and similar health related problems of an organism (such as a human or animal) comprising applying a composition of the present invention containing a medicament or other therapeutic component to the organism in need of treatment. In certain preferred embodiments, the step of applying the present composition comprises providing an MDI containing the composition of the present invention, including each of Pharmaceutical Compositions 1-10, and then discharging the present composition from the MDI.

The MDI metering valve size, that is, the size of the metering chamber, can vary within the scope hereof, but may be between 5 microliters (μL or mcl) and 200 microliters, between 25 microliters and 200 microliters, between 25 microliters and 150 microliters, between 25 microliters and 100 microliters, or between 25 microliters and 65 microliters.

In certain embodiments, typical compositions of the present disclosure include the fluticasone propionate in an amount of at least 0.04 milligram per actuation (mg/actuation) (40 microgram (μg) per actuation), 100, or at least 0.15 mg/actuation (150 μg/actuation), or at least 0.200 mg/actuation (200 μg/actuation), or at least 0.25 mg/actuation (250 μg/actuation), or at least 0.3 mg/actuation (300 μg/actuation). In certain embodiments, including each of the lower amounts identified above, typical compositions of the present disclosure include the fluticasone propionate in an amount of less than 0.5 mg/actuation (500 μg/actuation), or in an amount of less than 0.4 mg/actuation (400 μg/actuation).

The present invention includes methods of forming pharmaceutical compositions having improved stability, including each of Pharmaceutical Compositions 1-10, comprising forming a carrier comprising not less than 96% by weight of HFO-1234ze(E) and from greater than about 0.5% by weight to less than 5% by weight of ethanol, suspending in said carrier said fluticasone, preferably wherein said suspension has at least about great than 1 and less than about 6 mg/mL of fluticasone.

The present invention includes methods of forming pharmaceutical compositions having improved stability, including each of Pharmaceutical Compositions 1-10, comprising forming a carrier comprising not less than 96% by weight of HFO-1234ze(E) and from greater than about 0.5% by weight to less than 5% by weight of ethanol, suspending in said carrier an API comprising, consisting essentially of, or consisting of from 0.1 wt. % to about 0.5 wt. % fluticasone propionate.

### EXAMPLES

### Comparative1 Example 1: Stability of High and Low Dose Fluticasone Propionate Suspensions in HFO-1234ze(E) and in HFC-134a

¹The use of the term herein “comparative” with respect to an example is not intended to necessarily indicate that the example is in the prior art or representative of the prior art, and used for convenience to help illustrate one or more advantages of certain aspects or embodiments of the present invention.

Fluticasone propionate (API) was suspended in each of HFO-1234ze(E) and HFC-134a in amounts as indicated in Table C1 below to provide a nominal dose of 250 μg/actuation (for the formulations labeled below as HD (high dose)) and a nominal dose of 125 μg/actuation (for the formulations labeled below as LD (low dose)). The low dose formulation had about 1.9 mg/mL of fluticasone propionate, and the high dose formulation had about 3.8 mg/mL of fluticasone propionate. Each suspension was filled into glass vials with a 63-micro liter valve. Each formulation is tested for stability using the Turbiscan Stability Index (TSI). TSI is a test known to those skilled in the art as the cumulative sum of the change in light transmittance or backscattering throughout a sample of an API-containing formulation at series of times after the formulation has been formed into a suspension, as would occur for example after an MDI containing the sample has been shaken. In this test, higher values indicate a larger change from the baseline (T=0) and more instability. More stable suspensions have lower TSI values. The TSI is determined according to the following calculation:

$$TSI ( t ) = \frac{1}{{N}_{h}} {\sum }_{{t}_{i} = 1}^{{t}_{max}} {\sum }_{{z}_{i} = {z}_{min}}^{{z}_{max}} \vert BST ( {t}_{i} , {z}_{i} ) - BST ( {t}_{i - 1} , {z}_{i} ) \vert$$

where:
tₘₐₓ=measurement time point
zₘᵢₙ=lower scanning height limit
zₘₐₓ=upper scanning height limit
Nₕ=(zₘₐₓ−zₘᵢₙ)/Δh=number of height positions
BST is the signal (backscattering (BS) if transmission (T) is <0.2%, or T otherwise).

The test is performed by adding the fluticasone propionate in the indicated concentration into a clean, glass vial. The propellant (HFO-1234ze(E) or HFC-134a)) was then pressure filled through the valve to a final weight of 10 g.

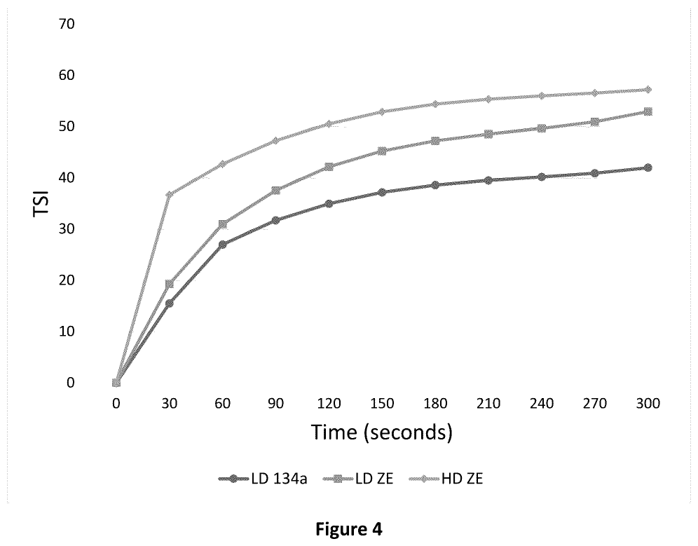

Vials were shaken vigorously for 10 seconds and then loaded into the Turbiscan Lab instrument (Formulaction, France). Samples were scanned at all heights every 30 seconds (or at intervals as otherwise indicated) over a 5-minute period at 25° C. The TSI was calculated from the bottom of the vial to the meniscus of each formulation. Mean values were calculated from sample heights of 8 to 14 mm. Peak thickness was measured from the bottom of the sample to 3 mm, with an absolute ΔBS threshold of 6%. For the purposes of this example, the results after the first 30 seconds are particularly important since those results represent the time closest to when the MDI would be used. The formulations and the results at 30 seconds are reported in Table ExC1 below, and all TSI results are illustrated in FIG. 4.

TABLE EXC1

Example No.

| | ExC1A | ExC1B | ExC1C |
|---|---|---|---|
| | (LD134a in | (LDZE in | (HDZE in |
| Components | FIG. C1) | FIG. C1) | FIG. C1) |
| HFC-1234ze(E), wt. % | 0 | 99.838 | 99.677 |
| HFC-134a, wt. % | 99.838 | 0 | 0 |
| API, wt. % | 0.162 | 0.162 | 0.323 |
| Total | 100 | 100 | 100 |
| TSI at 30 seconds | 15.5 | 26.96 | 31.68 |

As can been seen from the data presented in the chart above, the TSI after 30 seconds for HFC-134a (which represents a relatively low dose) is substantially superior to the result for HFO-1234ze(E) at that same low dose, and the magnitude of this superiority is even greater compared to the use of HFO-1234ze(E) at the high dose level.

### Comparative Example 2: Stability of High Dose Fluticasone Propionate Suspension in 95% HFO-1234ze(E) and 5% Ethanol

Comparative Example C1C is repeated, except the carrier consisted of HFO-1234ze with 5% of ethanol. The results at 30 seconds are reported in Table ExC2 below, and the result from Example C1C are reproduced for ease of comparison.

TABLE EXC2

Example No.

| Components | ExC1C | ExC2 |
|---|---|---|
| HFO-1234ze(E), wt. % | 99.677 | 94.693 |
| Ethanol, wt. % | 0 | 4.984 |
| API, wt. % | 0.323 | 0.323 |
| Total | 100 | 100 |
| TSI at 30 seconds | 31.68 | 39.83 |

This result indicates that the use of ethanol at a level of 5% substantially deteriorates the stability of the formulation at 30 seconds compared to the already inferior result of HFO-1234ze(E) alone compared to HFC-134a.

### Comparative Examples 3A-3C: Stability of High Dose Fluticasone Propionate Suspensions in About 95% HFO-1234ze(E), 5% Ethanol and Oleic Acid

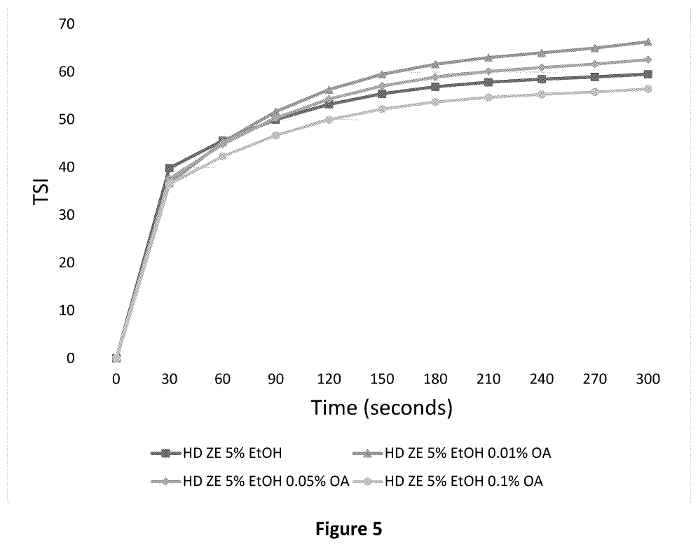

Comparative Example C2 is repeated, except oleic acid (hereinafter sometimes also referred to as “OA”) in amounts of 0.01%, 0.05% and 0.1% is used in the carrier, which otherwise consisted of 95% of HFO-1234ze and 5% of ethanol. The results at 30 seconds are reported in Table ExC3 below, and the result from the entire test (together with the results from Example C2 for ease of comparison) are shown in FIG. 5.

TABLE EXC3

Example No.

| Components | EXC2 | EXC3A | EXC3B | EXC3C |
|---|---|---|---|---|
| HFO-1234ze(E), | 94.693 | 94.683 | 94.643 | 94.603 |
| wt. % | | | | |
| Ethanol, wt. % | 4.984 | 4.984 | 4.984 | 4.984 |
| OA, wt. % | 0 | 0.01 | 0.05 | 0.1 |
| API, wt. % | 0.323 | 0.323 | 0.323 | 0.323 |
| Total | 100 | 100 | 100 | 100 |
| TSI at 30 | 39.83 | 36.69 | 37.58 | 36.49 |
| seconds | | | | |

These results indicate that the use of ethanol at 5% with the addition of OA in amounts of 0.01%, 0.05% and 0.1% causes an improvement in TSI stability that is relatively minor (e.g., 10 relative percent improvement or less in 30 second TSI stability for 5% ethanol formulations) and which does not adequately approach the stability using HFC-134a as the carrier.

### Examples 1A and 1B: Stability of High Dose Fluticasone Propionate Suspensions in HFO-1234ze(E) and 1% and 2.5% Ethanol

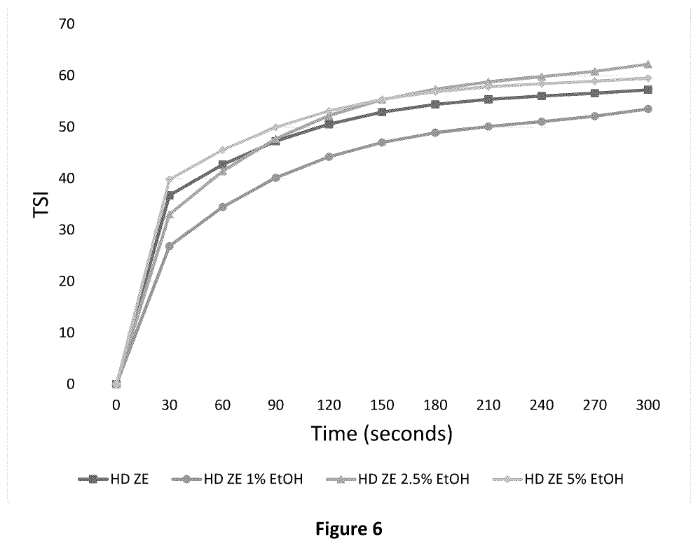

Fluticasone propionate (API) was suspended in mixtures of HFC-1234ze(E) and ethanol in amounts of 1% and 2.5% as indicated in Table E1 below in accordance with procedures described in Comparative Example 1, and the full set of results are provided in FIG. 6, together with the comparative high dose results from Comparative Examples 1 and 2 for ease of comparison.

TABLE E1

Example No.

| Components | Ex1A | Ex1B |
|---|---|---|
| HFO-1234ze(E), wt. % | 98.680 | 97.185 |
| Ethanol, wt. % | 0.997 | 2.492 |
| API, wt. % | 0.323 | 0.323 |
| Total | 100 | 100 |
| TSI at 30 seconds | 26.83 | 33.02 |

As can be seen from the data above, while the use of 2.5% ethanol and 97.5% of HFO-1234ze(E) represents an improvement in performance compared to the use HFO-1234ze(e) alone and the use of a 95/5 blend, the best result in this example is the use of carrier comprising 99% HFO-1234ze(E) and 1% of ethanol, which represents a relatively large and unexpected improvement in stability (e.g., a 35 relative percent improvement (i.e., reduction in TSI) compared to the formulation with 95% HFO-1234ze(E) and 5% ethanol).

### Examples 2A, 2B and 2C: Stability of High Dose Fluticasone Propionate Suspensions in HFO-1234ze(E) and Oleic Acid

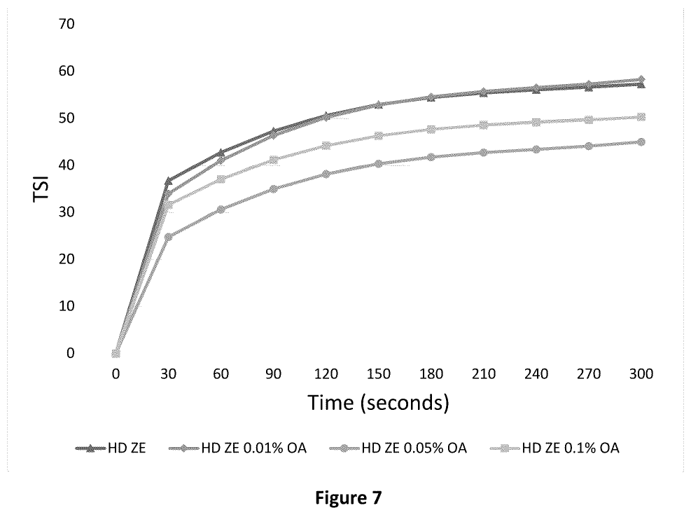

Fluticasone propionate (API) was suspended in HFC-1234ze(E) and oleic acid (hereinafter sometimes also referred to as “OA”) in amounts of 0.01%, 0.05% and 0.1% as indicated in Table E3 below in accordance with procedures described in Comparative Example 1, and the full set of results are provided illustrated FIG. 7, together with the comparative high dose results from Comparative Example C1C for ease of comparison.

TABLE E2

| | Example No. | | |
|---|---|---|---|
| | Ex2A | Ex2B | EX2C |
| Components | | | |
| Carrier Components | | | |
| HFO-1234ze(E), wt. % | 99.990 | 99.95 | 99.90 |
| Oleic Acid, wt. % | 0.01 | 0.05 | 0.10 |
| Total Carrier | 100.00 | 100.00 | 100.00 |
| Formulation | | | |
| Carrier, wt. % | 99.677 | 99.677 | 99.677 |
| API, wt. % | 0.323 | 0.323 | 0.323 |
| Total Formulation | 100 | 100 | 100 |
| TSI at 30 seconds | 33.92 | 24.72 | 31.57 |

As is seen from test result reported above, applicants have found that while the use of 0.01 wt %, 0.05 wt. % and 0.1 wt. % OA produces improvement in stability compared to a carrier consisting of HFO-1234ze(E) but without OA, the use of 0.05% of OA in HFO-1234ze(E) produces an unexpected and highly advantageous stability advantage compared to even to 0.01 wt % and 0.1 wt. % of OA in HFO-1234ze(E). In fact, the improvement shown by Example 2B to a TSI value of about 25 using a relative high dose of the API is unexpectedly able to bring the stability toward the stability shown for half the dose of the API in HFC-134a of about 16. This is an unexpected result.

### Comparative Example 3: Stability of High Dose Fluticasone Propionate Suspensions in About 99% HFO-1234ze(E), 1% Ethanol and Oleic Acid

Example E1A, which showed unexpected advantage over neat 1234ze(E) with the addition of 1% by weight of ethanol, is repeated except with the addition of oleic acid in amounts of 0.01 wt. % and 0.05 wt. %. The results are reported in Table ExC3 below, together with the comparative data from Ex1A.

TABLE EXC3

| | Example No. | | |
|---|---|---|---|
| Components | Ex1A | ExC3A | ExC3B |
| HFO-1234ze(E), | 98.680 | 98.670 | 98.630 |
| wt. % | | | |
| Ethanol, wt. % | 0.997 | 0.997 | 0.997 |
| Oleic Acid, wt. % | 0 | 0.010 | 0.050 |
| API, wt. % | 0.323 | 0.323 | 0.323 |
| Total | 100 | 100 | 100 |
| TSI at 25 seconds | 26.83 | 32.94 | 39.96 |

As can be seen from the data above, the addition of oleic acid in amounts of 0.01 wt. % and 0.05 wt. % results in a deterioration of stability performance when 1% ethanol is used with 99% of HFO-1234ze(E).

### Comparative Example 4: Stability of High Dose Fluticasone Propionate Suspensions in About 97.5% HFO-1234ze(E), 2.5% Ethanol and 0.05% Oleic Acid

Example E1B, which showed a slight advantage over neat 1234ze(E) at 30 seconds with the addition of 2.5% by weight of ethanol, is repeated except with the addition of oleic acid in an amount of 0.05 wt. %. The results are reported in Table ExC4 below, together with the comparative data from Ex1B.

TABLE EXC4

Example No.

| Components | Ex1B | ExC4 |
|---|---|---|
| HFO-1234ze(E), wt. % | 97.185 | 97.135 |
| Ethanol, wt. % | 2.492 | 2.492 |
| Oleic Acid, wt. % | 0 | 0.050 |
| API, wt. % | 0.323 | 0.323 |
| Total | 100 | 100 |
| TSI at 30 seconds | 33.02 | 37.66 |

As can be seen from the data above, the addition of oleic acid in amounts of 0.05 wt. % to this formulation also results in a deterioration of stability performance.

### Comparative Examples 5A-5B: Stability of High Dose Fluticasone Propionate Suspensions in HFO-1234ze(E) and 0.01% PEG (1000 MW) and in 99% HFO-1234ze(E), 1% Ethanol

Comparative Example C1C and Example 1A are repeated in a series of tests, except with the addition of polyethylene glycol having an average molecular weight of about 1000 (hereinafter sometimes referred to as “PEG1000”) in an amount of about 0.01% by weight in the carrier, except the time intervals for measuring the TSI index were 25 seconds. The results are reported in Table ExC5A below, together with Example C1C and Example 1A for ease of comparison.

TABLE EXC5A

| | Example No. | | | |
|---|---|---|---|---|
| Components | ExC1C | ExC5A | Ex1A | ExC5B |
| HFO-1234ze(E), wt. % | 99.677 | 99.667 | 98.680 | 98.670 |
| Ethanol, wt. % | 0 | 0 | 0.997 | 0.997 |
| PEG1000, wt. % | 0 | 0.010 | 0 | 0.010 |
| API, wt. % | 0.323 | 0.323 | 0.323 | 0.323 |
| Total | 100 | 100 | 100 | 100 |
| TSI at 25 seconds | 31.59 | 36.75 | 21.94 | 22.94 |

As can be seen from the data above, the addition PEG1000 in amounts of 0.01 wt. % to HFO-1234ze(E) results in a deterioration of 25 second TSI stability performance. With respect to a comparison to the inventive composition of the present invention represented by Ex1A (1% ethanol and 99% of 1234ze(E)) the carrier of this comparative example exhibits a slight deterioration in 25 second stability performance with the addition of 0.01 wt % of PEG1000.

### Example 3: Stability of High Dose Fluticasone Propionate Suspensions in About 99% HFO-1234ze(E), 1% Ethanol and 0.005% PEG1000

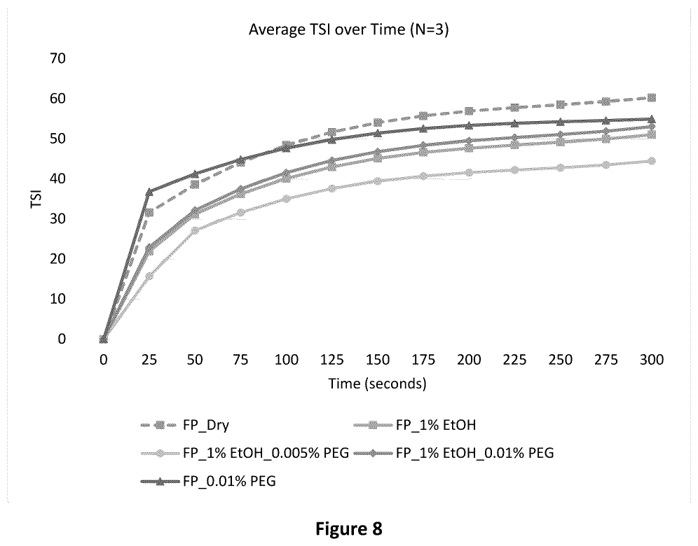

Comparative Example 5B is repeated, except the amount of PEG1000 in the formulation is 0.005 wt. %. The 25 second interval TSI stability testing on this formulation is reported in Table E3A below and in FIG. 8, together with the data reported in Comparative Example 5 for ease of comparison.

TABLE EX3A

| | Example No. | | | | |
|---|---|---|---|---|---|
| Components | ExC1C | ExC5A | Ex1A | ExC5B | Ex3 |
| HFO-1234ze(E), wt. % | 99.677 | 99.667 | 98.680 | 98.670 | 98.665 |
| Ethanol, wt. % | 0 | 0 | 0.997 | 0.997 | 0.997 |
| PEG1000, wt. % | 0 | 0.010 | 0 | 0.010 | 0.005 |
| API, wt. % | 0.323 | 0.323 | 0.323 | 0.323 | 0.323 |
| Total | 100 | 100 | 100 | 100 | 100 |
| TSI at 25 seconds | 31.59 | 36.75 | 21.94 | 22.94 | 15.74 |

As can be seen from the data above, the addition of PEG1000 in amounts of 0.005 wt. % to the formulation of this Example 3 (99% HFO-1234ze(E) and 1% ethanol) results in dramatic and unexpected advantage in formula stability compared to the results when the carrier consists of HFO-1234ze(E), namely, a two (2) times improvement (i.e., 50% reduction) in the 25 second TSI value. This result is important and unexpected, especially in view of the result from the Comparative Example indicating that a greater amount of PEG1000 (0.01) added to HFO-1234ze(E) worsens the 25 second TSI stability of the formulation compared to formulation in which the carrier consists of HFO-1234ze(E) (see ExC5A). It is additionally unexpected that the use of such a formulation would result in such an excellent stability (TSI=14.7) at a high dose (250 μg/actuation), which is superior to the stability that is achieved with the prior art HFC-134a (TSI=15.5) at a low dose (125 μg/actuation). This advantage could be used to help achieve MDI operation to deliver a full prescribed dose in one actuation of the MDI, as opposed to the current MDI operation with HFC-134a which requires two actuations of a low dose formulation to active the prescribed dose.

In addition, the dry formulation and the formulations containing PEG in this example were tested to determine the RSD %. In particular, three (3) actuations were made with each canister for each of these formulations, and the average delivered dose (“DD” in the table below) of those three actuations are reported below in Table Ex3B together with the root mean standard deviation (RSD %) for the three actuations.

TABLE EX3B

| | Example No. | | | |
|---|---|---|---|---|
| | ExC1C | ExC5A | ExC5B | Ex3 |
| Components | | | | |
| HFO-1234ze(E), wt. % | 99.677 | 99.667 | 98.670 | 98.665 |
| Ethanol, wt. % | 0 | 0 | 0.997 | 0.997 |
| PEG1000, wt. % | 0 | 0.010 | 0.010 | 0.005 |
| API, wt. % | 0.323 | 0.323 | 0.323 | 0.323 |
| Total | 100 | 100 | 100 | 100 |
| Spray Properties | | | | |
| DD, μg/actuation | 130 | 160 | 145 | 142 |
| RSD % | 23.45 | 18.46 | 10.24 | 12.93 |

As can be seen from the data above, the results for the formulations containing both 1% ethanol and PEG1000 produce RSD % values that are advantageously below about 15%, which is an additional unexpected result.

### Comparative Example 6: Stability of High Dose Fluticasone Propionate Suspensions in HFO-1234ze(E) and 0.005% PEG400 and in 99% HFO-1234ze(E), 1% Ethanol

Example 1A is repeated, except with the addition of polyethylene glycol having an average molecular weight of about 400 (hereinafter sometimes referred to as “PEG400”) in an amount of about 0.005% by weight in the carrier, and except the time intervals for measuring the TSI index were 25 seconds. The results are reported in Table ExC6 below, together with the results from Comparative Example C1C (100% HFO-1234ze(E)) and Example 1A for ease of comparison.

TABLE EXC6A

| | Example No. | | |
|---|---|---|---|
| Components | ExC1C | Ex1A | ExC5A |
| HFO-1234ze(E), wt. % | 99.677 | 98.680 | 98.665 |
| Ethanol, wt. % | 0 | 0.997 | 0.997 |
| PEG400, wt. % | 0 | 0 | 0.005 |
| API, wt. % | 0.323 | 0.323 | 0.323 |
| Total | 100 | 100 | 100 |
| TSI at 25 seconds | 31.59 | 21.94 | 33.4 |

As can be seen from the data above, the addition PEG400 in amounts of 0.005 wt. % to HFO-1234ze(E) and 1% ethanol results in a substantial deterioration of 25 second TSI stability performance compared to the 1% ethanol formulation but without the PEG400.

### Example 4: Stability of High Dose Fluticasone Propionate Suspensions in About 99% HFO-1234ze(E), 1% Ethanol and 0.01% Liquid PEG

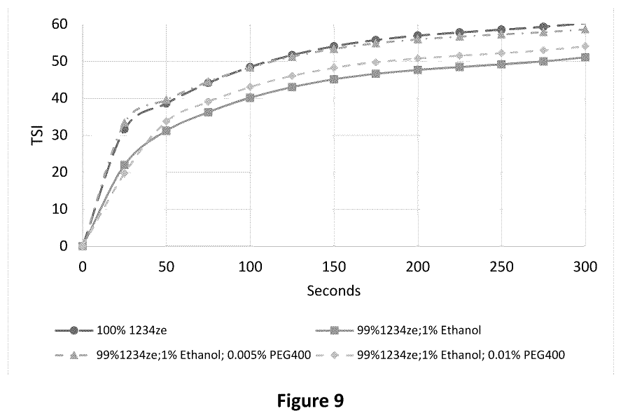

Comparative Example 6 is repeated, except the amount of PEG400 in the formulation is doubled to 0.01 wt. %. The 25 second interval TSI stability testing on this formulation is reported in Table Ex4 below and illustrated in FIG. 9, together with the data reported in Comparative Examples C1C, ExC6A and Example 1A for ease of comparison.

TABLE EX4

| | Example No. | | | |
|---|---|---|---|---|
| Components | ExC1C | Ex1A | ExC6 | Ex4 |
| HFO-1234ze(E), wt. % | 99.677 | 98.680 | 98.665 | 98.670 |
| Ethanol, wt. % | 0 | 0.997 | 0.997 | 0.997 |
| PEG400, wt. % | 0 | 0 | 0.005 | 0.010 |
| API, wt. % | 0.323 | 0.323 | 0.323 | 0.323 |
| Total | 100 | 100 | 100 | 100 |
| TSI at 25 seconds | 31.59 | 21.94 | 33.4 | 19.71 |

As can be seen from the data above, the addition of PEG400 in amounts of 0.01 wt. % to the formulation of this Example 4 (99% HFO-1234ze(E) and 1% ethanol) results in dramatic and unexpected advantage in formula stability compared to the results when the carrier consists of HFO-1234ze(E), namely, an almost two (2) times improvement (i.e., about 40% reduction) in the 25 second TSI value. This result is important and unexpected, especially in view of the result from the Comparative Example indicating that a lower amount of PEG400 (0.005 wt. %) added to HFO-1234ze(E) worsens the 25 second TSI stability of the formulation compared to formulation in which the carrier consists of HFO-1234ze(E) (see ExC5A).

What is claimed is:

1. A pharmaceutical composition comprising:
a. fine particles of fluticasone;
b. a carrier in which said fine particles of fluticasone are suspended, said carrier comprising: (i) from greater than 95% by weight to less than 99.5% by weight of HFO-1234ze(E); and (ii) from greater than 0.5% by weight to less than 5% by weight of ethanol.

2. The pharmaceutical composition of claim 1 further comprising polyethylene glycol (“PEG”).

3. The pharmaceutical composition of claim 2 wherein said PEG has an average molecular weight of from about 300 to about 1200.

4. The pharmaceutical composition of claim 2 wherein said PEG has an average molecular weight of about 1000.

5. The pharmaceutical composition of claim 2 wherein said PEG has an average molecular weight of from about 400.

6. The pharmaceutical composition of claim 1 wherein said carrier comprises: (i) from greater than 96% by weight to less than 99.5% by weight of HFO-1234ze(E); and (ii) from greater than 0.5% by weight to less than 4% by weight of ethanol.

7. The pharmaceutical composition of claim 1 wherein said carrier comprises: (i) from greater than 97% by weight to less than 99.5% by weight of HFO-1234ze(E); and (ii) from greater than 0.5% by weight to less than 3% by weight of ethanol.

8. The pharmaceutical composition of claim 1 wherein said carrier comprises: (i) from greater than 98% by weight to less than 99.5% by weight of HFO-1234ze(E); and (ii) from greater than 0.5% by weight to less than 2% by weight of ethanol.

9. The pharmaceutical composition of claim 1 wherein said carrier comprises: (i) about 99% by weight of HFO-1234ze(E); and (ii) about 1% by weight of ethanol.

10. The pharmaceutical composition of claim 1 wherein said pharmaceutical composition has a TSI stability at 25 seconds of 25 or less.

11. The pharmaceutical composition of claim 1 wherein said pharmaceutical composition has a TSI stability at 25 seconds of 20 or less.

12. The pharmaceutical composition of claim 1 wherein said pharmaceutical composition has a TSI stability at 25 seconds of 18 or less.

13. The pharmaceutical composition of claim 2 wherein said carrier comprises from greater than 0.001% by weight to less than 0.01% by weight of said PEG.

14. The pharmaceutical composition of claim 2 wherein said carrier comprises from greater than 0.001% by weight to less than 0.0075% by weight of said PEG.

15. The pharmaceutical composition of claim 2 wherein said carrier comprises from greater than 0.0025% by weight to less than 0.0075% by weight of said PEG.

16. The pharmaceutical composition of claim 2 wherein said carrier comprises from greater than 0.004% by weight to less than 0.006% by weight of said PEG.

17. The pharmaceutical composition of claim 1 wherein said fine particles of fluticasone comprise fluticasone propionate.

18. The pharmaceutical composition of claim 1 wherein said fine particles of fluticasone consist essentially of fluticasone propionate.

19. The pharmaceutical composition of any of claim 1 wherein said fine particles of fluticasone consist of fluticasone propionate.

20. An MDI containing a pharmaceutical composition of claim 1.
